(19) United States
(12) Patent Application Publication
(10) Pub. No.: US 2019/0038455 A1
(43) Pub. Date: Feb. 7, 2019

(54) Ambient Condition Resistant Body Mountable Thermal Coupling Devices

(71) Applicant: Verily Life Sciences LLC, Mountain View, CA (US)

(72) Inventors: Roxana Heitz, Mountain View, CA (US); William Biederman, San Francisco, CA (US); Sean Frick, San Francisco, CA (US)

(73) Assignee: Verily Life Sciences LLC, Mountain View, CA (US)

(21) Appl. No.: 15/665,991

(22) Filed: Aug. 1, 2017

(51) Int. Cl. A61F 7/00; A61F 7/02

(52) U.S. Cl. CPC A61F 7/007; A61F 2007/0096; A61F 2007/0226; A61F 7/02

(57) ABSTRACT

The technology described herein relates to body mountable thermal coupling devices and, more specifically, to body mountable thermal coupling apparatuses with resistance to varying ambient conditions. In some implementations, a body mountable thermal coupling apparatus is disclosed. The apparatus includes a bio-compatible thermally conductive metal disc, a substrate, a thermal sensor, an enclosure, and an adhesive patch. The described apparatus facilitates enhanced thermal coupling between a heat source, e.g., human skin, and a thermal (or temperature) sensor.

### BACKGROUND

In recent years there has been a growing interest for active medical technologies that leverage the increasing computational power of portable computers, smartphones, and tablets. For example, body mountable thermal coupling devices (or patches) that measure and track the temperature of a user's body currently exist. These devices can, and often are, worn for lengthy periods of time, e.g., a 24-hour period.

### SUMMARY

Examples discussed herein relate to body mountable thermal coupling devices and, more specifically, to body mountable thermal coupling apparatuses with resistance to varying ambient conditions. In an implementation, a body mountable thermal coupling apparatus is disclosed. The apparatus includes a bio-compatible thermally conductive metal disc embedded in or otherwise attached to an enclosure, a substrate, a thermal sensor, an enclosure, and an adhesive patch. The bio-compatible thermally conductive metal disc has a proximal surface for thermally coupling with the skin of a user. The substrate has a proximal surface with an exposed conductive pad thermally coupled to a distal surface of the metal disc. The substrate includes one or more through-substrate vias filled with thermally conductive material.

The thermal sensor is disposed on a distal surface of the substrate and is thermally coupled to one or more through-substrate vias. The enclosure includes distal and proximal portions for encasing the substrate. The adhesive patch is affixed to a proximal surface of the proximal (or bottom) portion of the enclosure. The adhesive patch includes an opening (or cutout) for the metal disc and a bio-compatible adhesive on the proximal surface for removably attaching the apparatus to the skin of the user.

This Overview is provided to introduce a selection of concepts in a simplified form that are further described below in the Technical Disclosure. It may be understood that this Overview is not intended to identify key features or essential features of the claimed subject matter, nor is it intended to be used to limit the scope of the claimed subject matter.

### BRIEF DESCRIPTION OF THE DRAWINGS

A detailed description is set forth and will be rendered by reference to specific examples thereof which are illustrated in the appended drawings. Understanding that these drawings depict only typical examples and are not therefore to be considered to be limiting of its scope, implementations will be described and explained with additional specificity and detail through the use of the accompanying drawings.

FIG. 1 depicts a diagram illustrating an example operational architecture for operating an ambient condition resistant body mountable thermal coupling device, according to some implementations.

FIG. 2A illustrates a top view of the ambient condition resistant body mountable thermal coupling device with an attached enclosure, according to some implementations.

FIG. 2B illustrates a top view of the ambient condition resistant body mountable thermal coupling device with a distal portion of enclosure removed, according to some implementations.

FIG. 2C illustrates a bottom view of the ambient condition resistant body mountable thermal coupling device, according to some implementations.

FIG. 3A illustrates a cross-sectional side view of the ambient condition resistant body mountable thermal coupling device with attached distal and proximal enclosure portions for encasing a substrate, according to some implementations.

FIG. 3B illustrates an exploded cross-sectional side view of the ambient condition resistant body mountable thermal coupling device, according to some implementations.

FIG. 3C illustrates an exploded perspective view of the ambient condition resistant body mountable thermal coupling device, according to some implementations.

FIG. 4 illustrates a side view of an example substrate in surface mount packaging with a thermal sensor mounted in the package, according to some implementations.

FIG. 5 illustrates an example ambient condition resistant body mountable thermal coupling device, according to some implementations.

FIG. 6 illustrates an example ambient condition resistant body mountable thermal coupling device, according to some implementations.

FIG. 7 depicts a block diagram illustrating an example operational architecture for operating an ambient condition resistant body mountable thermal coupling device, according to some implementations.

The drawings have not necessarily been drawn to scale. Similarly, some components and/or operations may be separated into different blocks or combined into a single block for the purposes of discussion of some of the embodiments of the present technology. Moreover, while the technology is amenable to various modifications and alternative forms, specific embodiments have been shown by way of example in the drawings and are described in detail below. The intention, however, is not to limit the technology to the particular embodiments described. On the contrary, the technology is intended to cover all modifications, equivalents, and alternatives falling within the scope of the technology as defined by the appended claims.

### DETAILED DESCRIPTION

Examples are discussed in detail below. While specific implementations are discussed, it should be understood that this is done for illustration purposes only. A person skilled in the relevant art will recognize that other components and configurations may be used without parting from the spirit and scope of the subject matter of this disclosure. The implementations may include machine-implemented methods, computing devices, or computer readable medium.

Body mountable thermal coupling devices (or patches) that measure and track the temperature of a user's body can, and often are, worn for lengthy periods of time, e.g., 24-hour periods or longer. With increased length of use comes an increased likelihood that ambient conditions vary during usage. However, existing body mountable devices cannot accurately and reliably estimate core body temperature of a user in environments with varying ambient conditions. For example, variations in ambient temperature, ambient humidity, or even ambient pressure can result in inaccurate core body temperature estimates when using existing body mountable thermal coupling devices (or patches).

Additionally, the existing body mountable thermal coupling devices (or patches) use high accuracy thermistors to measure core body temperature of a user. Unfortunately, the high accuracy thermistors are relatively expensive in terms of cost and can be difficult to place within an apparatus or device. For example, standard thermometers fit the thermistor in a “tip” of the device and package the electronics in a “body” of the device.

The technology described herein is directed to body mountable thermal coupling devices and, more specifically, to body mountable thermal coupling apparatuses with resistance to varying ambient conditions. In some implementations, component stack-ups for ambient condition resistant body mountable thermal coupling apparatuses are described that facilitate thermal coupling between heat from a human body and a thermal (or temperature) sensor. The body mountable thermal coupling apparatuses facilitate proper and reliable thermal coupling without jeopardizing moisture resistance of the electronics enclosure, etc.

In some implementations, heat from the human body is coupled to a thermal sensor on a silicon die through a stack-up including a gold-plated brass disc, a printed circuit board (PCB), and an adhesive patch. The gold-plated brass disc is built into an enclosure to ensure thermal coupling with the skin of a user and with the proximal side of the PCB. The disc can be inserted, molded or glued into the enclosure. As discussed herein, the proximal (or bottom) side or portion of a component is the side or portion that is body facing. Likewise, the distal (or top) side or portion is the opposing side or portion, i.e., not body facing.

The brass disc is thermally coupled with the exposed copper pad on the proximal side of the PCB. In some implementations, thermal grease at the interface ensures uniform contact and improved thermal conductivity. A through-board via filled with conductive epoxy or metal carries heat to the distal side of the PCB where the temperature sensor is mounted. When the sensor is mounted in a particular type of package, e.g., wafer-level chip scale package, thermally conductive underfill can be used to improve the thermal conductivity. The apparatus can be attached to the skin of a user with an adhesive patch including an opening (or cutout) for the brass disc.

As noted above, existing body mountable thermal coupling devices (or patches) use relatively expensive thermistors to sense or measure temperature. Among other benefits, the stack-up described herein facilitates use of silicon thermal sensors for temperature sensing within a device. The silicon thermal sensors are less expensive, easier to place within a device and provide highly accurate thermal readings.

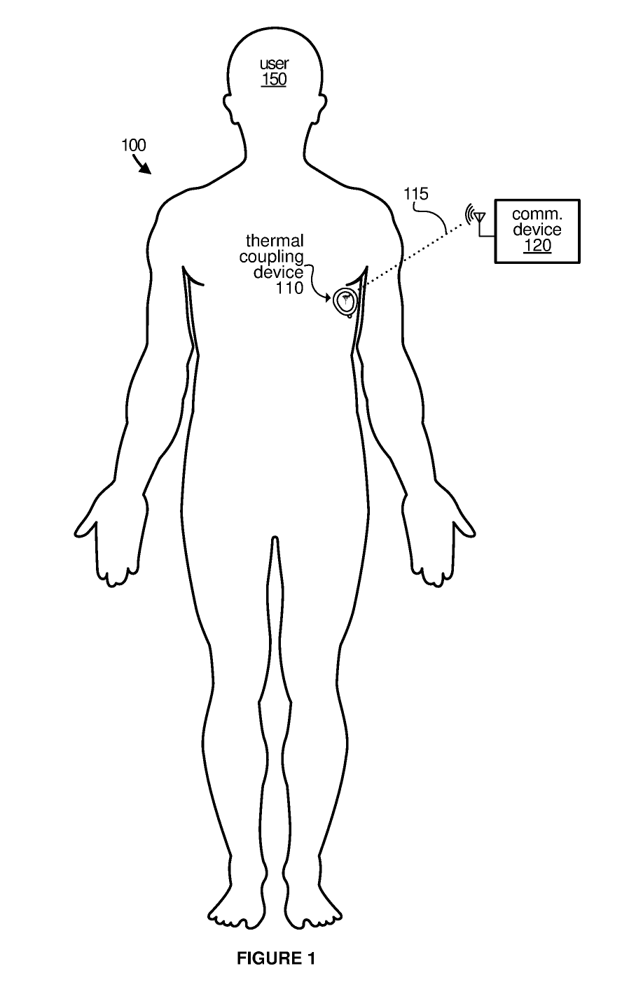

FIG. 1 depicts a diagram illustrating an example operational architecture 100 for operating an ambient condition resistant body mountable thermal coupling device 110, according to some implementations. As shown in the example of FIG. 1, the thermal coupling device 110 is affixed near the armpit of user 150.

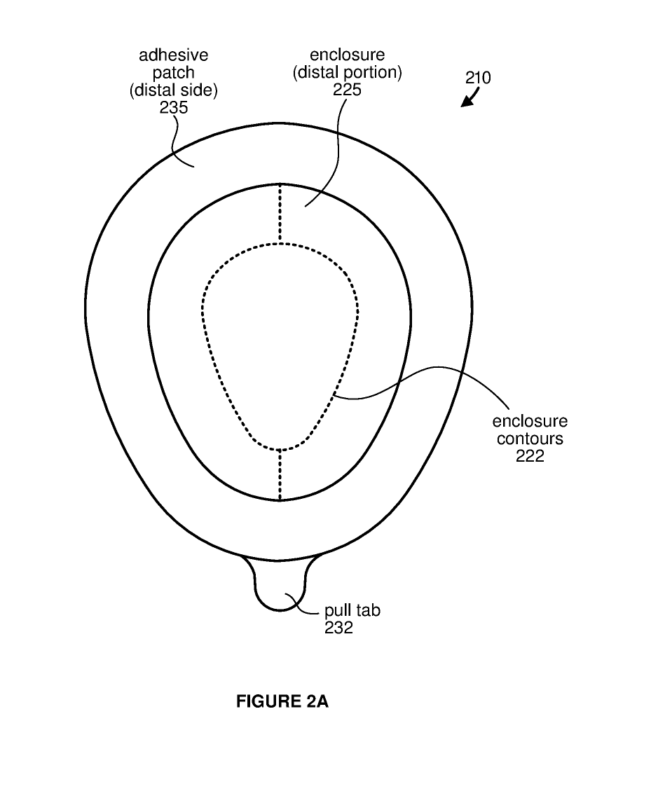

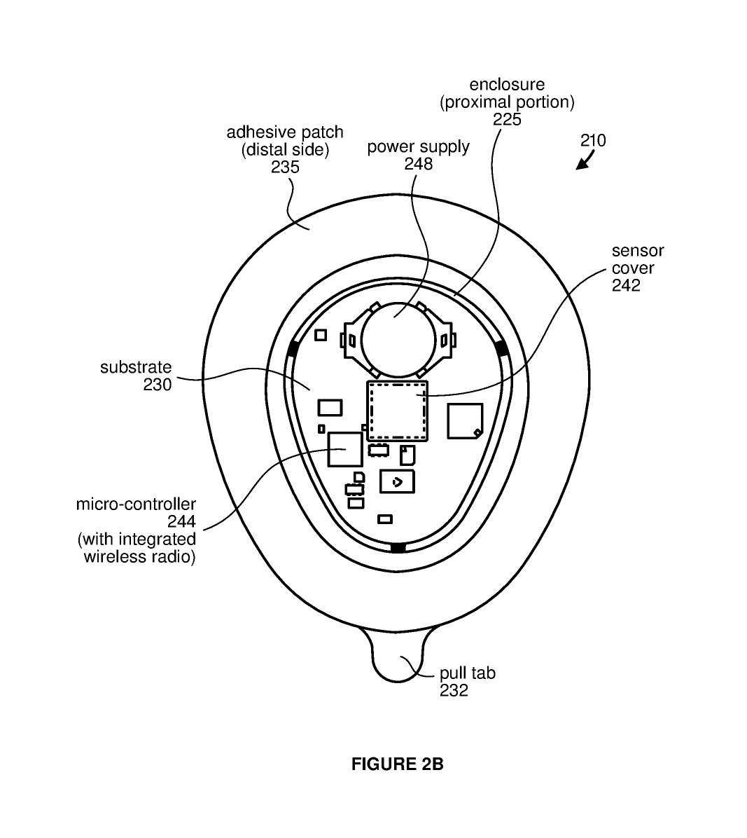

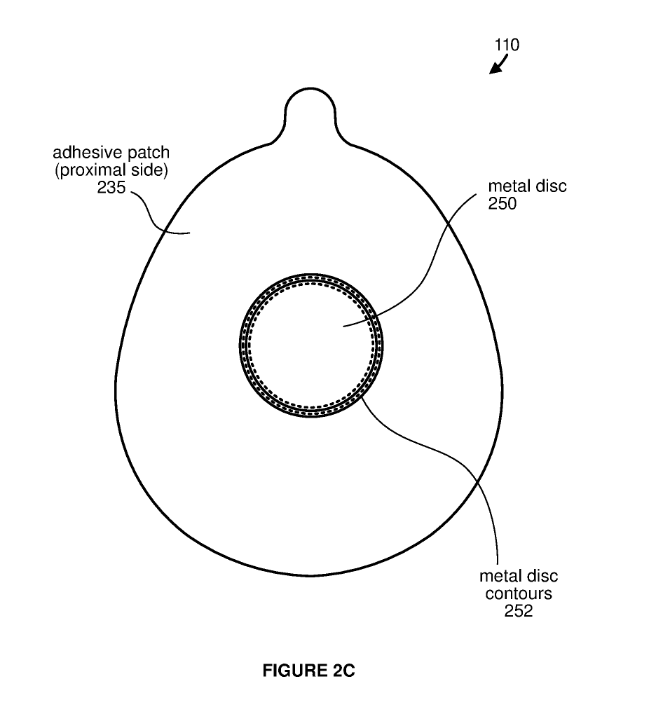

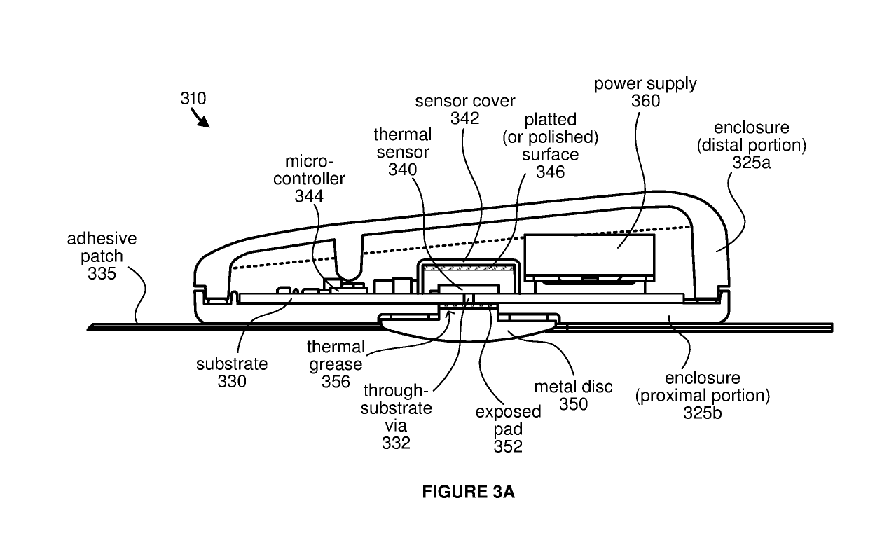

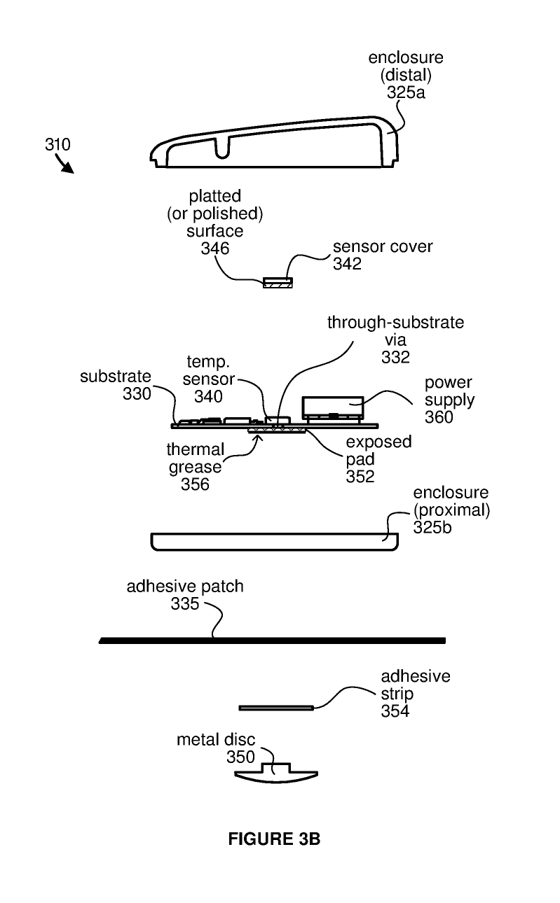

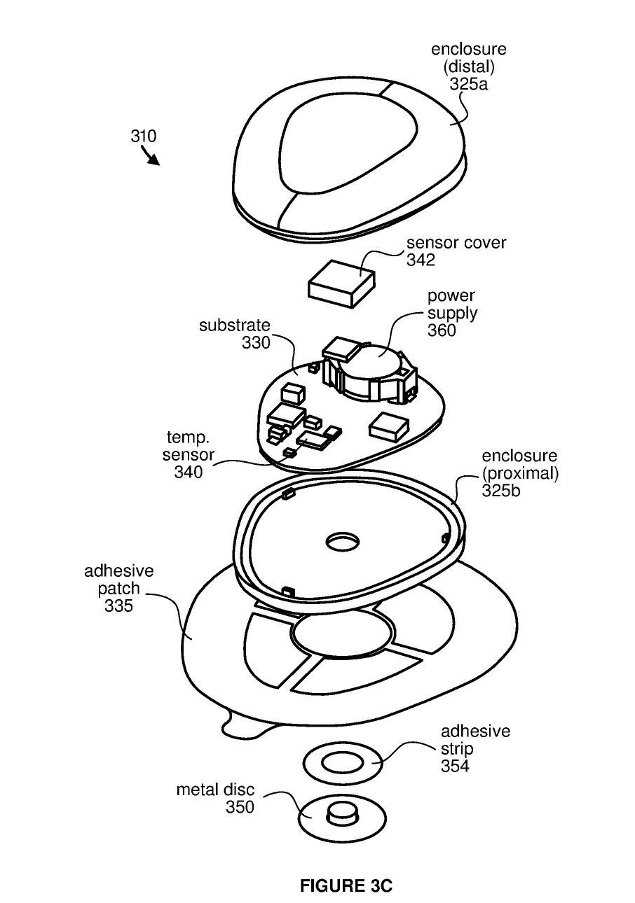

In operation, the thermal coupling device 110 estimates the core body temperature of user 150. Among other benefits, the thermal coupling device 110 is ambient condition resistant and, thus, can be worn and accurately estimate core body temperature of the user 150 for extended periods of time regardless of changes to ambient conditions. Example ambient condition resistant body mountable thermal coupling devices are shown and discussed in greater detail with reference to FIGS. 2A-2C and FIGS. 3A-3C.

FIGS. 2A-2C depict various views of an example ambient condition resistant body mountable thermal coupling device 210, according to some implementations. The ambient condition resistant body mountable thermal coupling device 210 can be ambient condition resistant body mountable thermal coupling device 110 of FIG. 1, although alternative configurations are possible.

Referring first to FIG. 2A, the example of FIG. 2A illustrates a top view of the ambient condition resistant body mountable thermal coupling device 210 with an attached enclosure 225. Enclosure 225 can be any bio-compatible housing or casing configured to shield components of the ambient condition resistant body mountable thermal coupling device 210. Enclosure 225 can be constructed of various materials, including plastics, rubbers, etc., that provide durability and moisture resistance.

As shown in the example of FIG. 2A, body mountable thermal coupling device 210 includes adhesive patch 235. The adhesive patch 235 can be constructed of various materials, including plastics, or natural or synthetic fabrics. The materials are chosen for, among other factors, durability and breathability. In some implementations, the adhesive patch 235 includes a bio-compatible adhesive on the proximal surface for removably attaching the apparatus to the skin of the user, e.g., user 150 of FIG. 1. Although not shown, a film or paper can be pulled away from the proximal surface of adhesive patch 235 prior to applying the device or apparatus to the skin of the user.

The examples of FIGS. 2A-2C also illustrate a pull tab 232. Unlike the rest of the proximal surface of the adhesive patch, the pull tab 232 does not include an adhesive. This allows a user to easily grab the pull tab 232 to remove the body mountable thermal coupling device 210.

Referring next to FIG. 2B, FIG. 2B illustrates a top view of the ambient condition resistant body mountable thermal coupling device 210 with a distal portion of enclosure 225 removed. As shown in the example of FIG. 2B, substrate 230 includes a thermal sensor (not shown) covered by a sensor cover 242, a microcontroller 244 (with an embedded wireless radio) and a power supply 248. Although located on the same chip in the example of FIG. 2B, it is appreciated that the wireless radio and the microcontroller 244 can be a multi-chip solution. In some implementations, substrate 230 can be a circuit board or printed circuit board (PCB). Additional or fewer components are possible.

Referring next to FIG. 2C, FIG. 2C illustrates a bottom view of ambient condition resistant body mountable thermal coupling device 210. As illustrated in the example of FIG. 2C, a bio-compatible thermally conductive metal disc 250 is shown. The thermally conductive metal disc 250 has a proximal surface adapted for thermal coupling with the skin of a user. The adhesive patch 235 includes a cutout (or opening) for the distal side of metal disc 250. Importantly, the interface where the metal disc 250 protrudes through the adhesive patch 235 is water and moisture resistant.

FIG. 3A-3C depict various views of an example ambient condition resistant body mountable thermal coupling device 310, according to some implementations. The body mountable thermal coupling device 310 can be body mountable thermal coupling device 110 of FIG. 1, although alternative configurations are possible.

Referring first to FIG. 3A, FIG. 3A illustrates a cross-sectional side view of the body mountable thermal coupling device 310 with attached distal and proximal enclosure portions 325a and 325b , respectively, for encasing substrate 330. As discussed above, enclosure 325 can be constructed of various materials, including plastics, rubbers, etc., designed for moisture resistance, including combinations or variations thereof.

As shown in the example of FIG. 3A, the body mountable thermal coupling device 310 includes a bio-compatible thermally conductive metal disc 350. Bio-compatible thermally conductive metal disc 350 can be any conductive material. In some implementations, bio-compatible thermally conductive metal disc 350 is a gold-plated brass disc that facilitates thermal coupling with the skin of the user. The proximal surface of the conductive metal disc 350 is adapted for proper and reliable thermal coupling. In the example of FIG. 3A, the proximal surface of the conductive metal disc 350 is convex to establish close contact with the skin of the user for a proper and reliable thermal couple.

The ambient condition resistant body mountable thermal coupling device 310 further includes a substrate 330 having a proximal surface with an exposed conductive pad 352 thermally coupled to a distal surface of the conductive metal disc 350. The exposed conductive pad 352 can be any conductive surface such as, for example, a copper pad. Additionally, in some implementations, a layer of thermal grease 356 is disposed at the interface between the exposed conductive pad 352 and the distal surface of the conductive metal disc 350 to increase the accuracy of the thermal coupling and reduce loss.

As shown, substrate 330 includes one or more through-substrate vias 332 filled with conductive materials that carry heat from the exposed pad 352 to thermal sensor 340. Thermal sensor 340 can be any sensor that senses temperature, e.g., one or more thermocouples. The sensor cover 342 is disposed on top of (or over) thermal sensor 340 to provide ambient temperature insulation and otherwise reduce ambient thermal coupling by thermal sensor 340. The ambient heat can include, for example, heat from the top of the device, heat from other electronics disposed on substrate 330, etc. The sensor cover 342 can be designed to include a space (or gap) between the sensor cover 342 and the thermal sensor 340 to provide additional insulation. The space can be filled with air or another thermally insulating material such as, for example, foam, etc.

In some implementations, the sensor cover 342 is polished or plated 346 to provide additional insulation. The polish or plating can be on the interior surface of the sensor cover 342 and/or the exterior surface. Although not shown, the distal enclosure portion 325a can alternatively or additionally be polished or plated on the interior and/or the exterior surface to provide insulation.

In some implementations, the substrate 330 includes a microcontroller 344 with an integrated wireless transmitter and a power supply 360. The microcontroller 344 is configured to estimate core body temperature of a user based, at least in part, on the temperature measurements of thermal sensor 340. Additionally, the microcontroller 344 uses input from other sensors (not shown) in addition to the temperature measurements from thermal sensor 340 to compensate and estimate core body temperature of the user.

As shown in the example of FIG. 3A, the enclosure includes a distal portion 325a and a proximal portion 325b . When the portions are connected, the substrate 330 is encased (or protected). As shown in the example of FIGS. 3A-3C, an adhesive patch 335 is affixed to a proximal surface of the proximal portion of the enclosure 325b . The adhesive patch 335 includes an opening for metal disc 350 and a bio-compatible adhesive on the proximal surface for removably attaching the apparatus to the skin of the user.

Referring next to FIG. 3B, FIG. 3B illustrates an exploded cross-sectional side view of the body mountable thermal coupling device 310. The exploded cross-sectional side view illustrates the components of FIG. 3A. As shown, FIG. 3B also includes adhesive strip 354. In some implementations, adhesive strip 354 is designed to, among other features, attach the metal disc 350 to the adhesive patch 335. The adhesive strip 354 can be a double-sided adhesive strip with an opening for the metal disc 350. The adhesive strip 354 attaches the metal disc 350 to the adhesive patch 355 and thereby to the proximal portion of enclosure 325b . In some implementations, the adhesive strip 354 can be a molded insert that connectably attaches the metal disc 350 to the proximal portion of enclosure 325b.

Referring next to FIG. 3C, FIG. 3C illustrates an exploded perspective view of the ambient condition resistant body mountable thermal coupling device 310. The exploded perspective view illustrates the components of FIGS. 3A and 3B. Additionally, the example of FIG. 3C illustrates a mushroom-shaped conductive metal disc 350 with a stem on the distal side that is thermally coupled to the proximal surface of an exposed conductive pad (not shown) disposed on the proximal surface of the substrate 330.

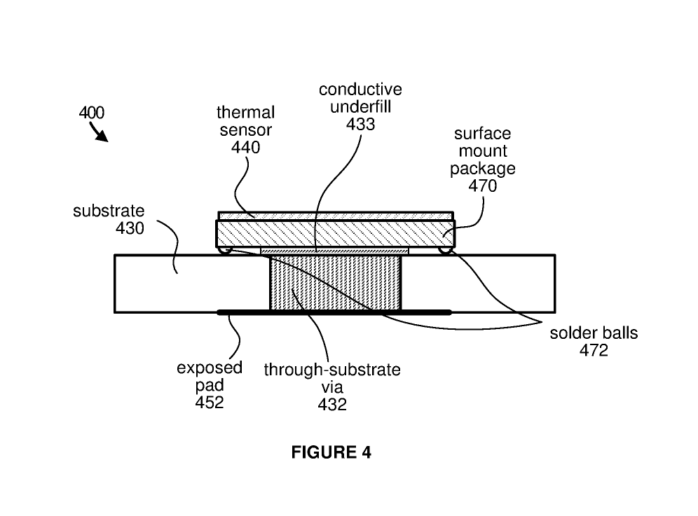

FIG. 4 illustrates a side view of an example substrate 430 with a surface mount ball grid array (B GA) packaging 470 having a thermal sensor 440 mounted in the package, according to some implementations. More specifically, as shown in the example of FIG. 4, the substrate 430 is a printed circuit board (PCB) and the thermal sensor 440 is an integrated circuit packaged in the surface-mount packaging 470 which is soldered to the substrate 430 with one or more solder balls 472. To improve thermally coupling, thermally conductive underfill 433 is provided to carry heat.

In operation, the thermally coupled heat at exposed pad 452 is carried through-substrate via 432 and thermally conductive underfill 433 to the thermal sensor 440. Although not shown in the example of FIG. 4, multiple through-substrate vias 432 can be included. For example, if the surface mount package 470 is a quad-flat no-leads (QFN) package that has a bottom pad, then multiple through-substrate vias 432 that do not overlap the bottom pad can be used to carry the heat through the substrate 430. Combinations and variations are possible.

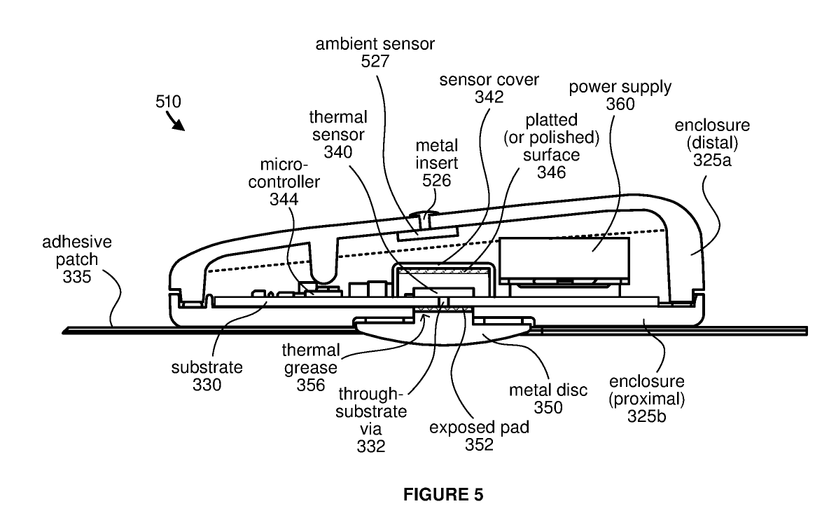

FIG. 5 illustrates an example ambient condition resistant body mountable thermal coupling device 510, according to some implementations. The ambient condition resistant body mountable thermal coupling device 510 can be ambient condition resistant body mountable thermal coupling device 110 of FIG. 1, although alternative configurations are possible.

The ambient condition resistant body mountable thermal coupling device 510 includes many of the components of the ambient condition resistant body mountable thermal coupling device 310 of FIGS. 3A-3C, but also includes an additional thermal sensor, i.e., ambient sensor 527 that senses ambient temperature. As shown in the example of FIG. 5, the ambient sensor 527 is mounted to the proximal surface of the distal portion of enclosure 325a and is thermally coupled to metal insert 526. The metal insert 526 thermally coupled to external ambient heat. As shown, the metal insert 526 is attached or otherwise embedded into the distal portion of enclosure 325a with the distal portion of enclosure 325a including an opening for the metal insert 526.

In some implementations, the ambient sensor 527 can be thermally coupled to metal insert 526 using mechanisms similar to the mechanisms used to thermally couple metal disc 350 and thermal sensor 340. For example, thermal grease may be applied at the interface between the ambient sensor 527 and the metal insert 526. It is appreciated that the ambient sensor 527 can be mounted in a variety of locations to improve knowledge of ambient temperature. For example, among other locations, the ambient sensor 527 can be mounted on substrate 330, sensor cover 342, or externally on the distal portion of enclosure 325a . Although not shown in the example of FIG. 5, one or more vias can be included to thermally couple the ambient sensor 527 to the metal insert 526, when necessary.

As discussed herein, the ambient sensor 527 senses the ambient temperature and provides this information to microcontroller 344. In some implementations, microcontroller 344 uses the ambient temperature as input to compensation algorithms when estimating core body temperature of the user. As discussed herein, the microcontroller 344 can estimate core body temperature of a user based, at least in part, on the temperature measurements of thermal sensor 340 and ambient sensor 527. Additionally, microcontroller 344 can use input from other sensors (not shown) compensate when estimating core body temperature of a user.

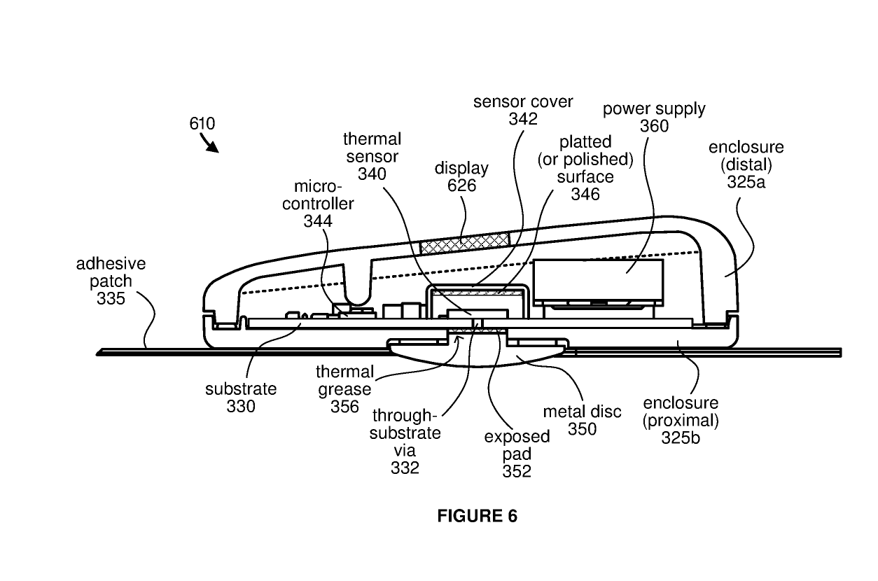

FIG. 6 illustrates an example ambient condition resistant body mountable thermal coupling device 610, according to some implementations. The ambient condition resistant body mountable thermal coupling device 610 can be ambient condition resistant body mountable thermal coupling device 110 of FIG. 1, although alternative configurations are possible. The ambient condition resistant body mountable thermal coupling device 610 includes many of the components of the ambient condition resistant body mountable thermal coupling device 310 of FIGS. 3A-3C, but also includes a display 626.

In some implementations, display 626 can illustrate the estimated core body temperature of the user. Display 626 can be included in addition to, or in lieu of, a wireless transmitter that transmits the estimated core body temperature of a user to a remote communication device, e.g., communication device 120 of FIG. 1, as discussed herein.

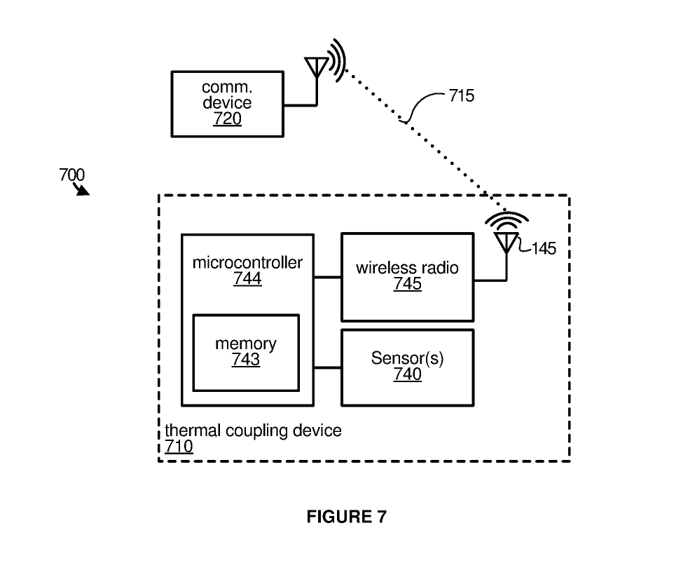

FIG. 7 depicts a block diagram illustrating an example operational architecture 700 for operating an ambient condition resistant body mountable thermal coupling device 710, according to some implementations. More specifically, the example of FIG. 7 illustrates example components of the thermal coupling device 710.

As shown in the example of FIG. 7, operational architecture 700 includes communication device 720 and thermal coupling device 710. The thermal coupling device 710 includes a microcontroller 705, a wireless radio 707 and one or more sensor(s) 740. Although shown as discrete components, one or more components can be combined. For example, wireless radio 707 can be embedded in a microcontroller system-on-a-chip (SoC).

In some implementations, microcontroller 744 executing program code, e.g., a compensation algorithm, from memory 743, samples the one or more sensors 740 and estimates a core body temperature of a user based on the samples. As discussed herein, the one or more sensors 740 can include one or more thermal sensors, humidity sensors, pressure sensors, etc.

The microcontroller 744 can be a small computer or other circuitry that retrieves and executes software from memory 743. The microcontroller 744 may be implemented within a single device or system-on-a-chip (SoC) or may be distributed across multiple processing devices that cooperate in executing program instructions. As shown in the example of FIG. 7, the microcontroller 744 is operatively or communicatively coupled with a wireless radio 745. Memory 743 can include program memory and data memory.

The functional block diagrams, operational scenarios and sequences, and flow diagrams provided in the Figures are representative of exemplary systems, environments, and methodologies for performing novel aspects of the disclosure. While, for purposes of simplicity of explanation, methods included herein may be in the form of a functional diagram, operational scenario or sequence, or flow diagram, and may be described as a series of acts, it is to be understood and appreciated that the methods are not limited by the order of acts, as some acts may, in accordance therewith, occur in a different order and/or concurrently with other acts from that shown and described herein. For example, those skilled in the art will understand and appreciate that a method could alternatively be represented as a series of interrelated states or events, such as in a state diagram. Moreover, not all acts illustrated in a methodology may be required for a novel implementation.

The descriptions and figures included herein depict specific implementations to teach those skilled in the art how to make and use the best option. For the purpose of teaching inventive principles, some conventional aspects have been simplified or omitted. Those skilled in the art will appreciate variations from these implementations that fall within the scope of the invention. Those skilled in the art will also appreciate that the features described above can be combined in various ways to form multiple implementations. As a result, the invention is not limited to the specific implementations described above, but only by the claims and their equivalents.

What is claimed is:

1. A body mountable thermal coupling apparatus comprising:
a bio-compatible thermally conductive metal disc having a proximal surface for thermal coupling with skin of a user;
a substrate having a proximal surface with an exposed conductive pad thermally coupled to a distal surface of the metal disc, the substrate including one or more through-substrate vias filled with thermally conductive material;
a thermal sensor disposed on a distal surface of the substrate and thermally coupled to the one or more through-substrate vias;
an enclosure including a proximal portion and a distal portion that encase the substrate; and
an adhesive patch affixed to the proximal surface of the enclosure, the adhesive patch including an opening for the metal disc, and a bio-compatible adhesive on a proximal surface for removably attaching the thermal coupling apparatus to the skin of the user.

2. The body mountable thermal coupling apparatus of claim 1, further comprising:
a layer of thermal grease disposed at an interface between the exposed conductive pad and the distal surface of the metal disc.

3. The body mountable thermal coupling apparatus of claim 1, wherein the thermally conductive material comprises a conductive epoxy or metal.

4. The body mountable thermal coupling apparatus of claim 1, wherein the substrate comprises a printed circuit board (PCB).

5. The body mountable thermal coupling apparatus of claim 4, further comprising:
conductive underfill disposed in gaps between a surface-mount packaging and the PCB,
wherein the thermal sensor comprises an integrated circuit that is packaged in the surface-mount packaging.

6. The body mountable thermal coupling apparatus of claim 1, further comprising:
a thermal sensor cover disposed over the thermal sensor.

7. The body mountable thermal coupling apparatus of claim 6, wherein the thermal sensor cover is plated or polished.

8. The body mountable thermal coupling apparatus of claim 7, wherein thermal sensor cover includes an air gap between the thermal sensor cover and the thermal sensor.

9. The body mountable thermal coupling apparatus of claim 1, wherein distal or proximal portions of the enclosure are plated or polished.

10. The body mountable thermal coupling apparatus of claim 1, further comprising:
a wireless transmitter configured to transmit temperature related information to a receiving communication device.

11. The body mountable thermal coupling apparatus of claim 1, further comprising:
a second thermal sensor that senses ambient temperature, the second thermal sensor disposed on a proximal surface of the distal portion of the enclosure; and
a microcontroller configured to estimate core body temperature of the user based, at least in part, on output of the thermal sensor and the ambient temperature.

12. The body mountable thermal coupling apparatus of claim 1, wherein the metal disc is mushroom-shaped with a stem that is thermally coupled with the exposed conductive pad on the proximal surface of the substrate.

13. The body mountable thermal coupling apparatus of claim 1, wherein the proximal surface of the metal disc is convex.

14. The body mountable thermal coupling apparatus of claim 1, wherein the metal disc comprises a gold-plated brass disc.

15. The body mountable thermal coupling apparatus of claim 1, wherein the exposed conductive pad on the proximal surface of the substrate comprises a copper pad.

16. The body mountable thermal coupling apparatus of claim 1, further comprising:
a display that graphically indicates an estimated core body temperature of the user.

17. A body mountable thermal coupling device comprising:
a bio-compatible thermally conductive metal disc having a proximal surface adapted for thermal coupling with skin of a user;
a substrate having a proximal surface with an exposed conductive pad thermally coupled to a distal surface of the metal disc with a layer of thermal grease, the substrate including one or more through-substrate vias filled with thermally conductive material;
a thermal sensor that senses a temperature of the skin of the user, the thermal sensor disposed on a distal surface of the substrate and thermally coupled to the exposed conductive pad over the one or more through-substrate vias; and
an adhesive patch adapted for removably attaching the body mountable thermal coupling device to the skin of the user.

18. The body mountable thermal coupling device of claim 17, further comprising a pull tab affixed to the adhesive patch.

19. The body mountable thermal coupling device of claim 17, wherein the substrate comprises a printed circuit board (PCB), the device further comprising:
conductive underfill disposed in gaps between a surface-mount packaging and the PCB, wherein the thermal sensor comprises an integrated circuit that is packaged in the surface-mount packaging.

20. A body mountable thermal coupling device comprising:
a bio-compatible thermally conductive metal disc having a proximal surface adapted for thermal coupling with skin of a user;
a thermal sensor disposed on a substrate and thermally coupled to the metal disc;
a thermal sensor cover disposed over the thermal sensor;
an enclosure including a distal portion and a proximal portion that encase the substrate; and
an adhesive patch affixed to a proximal surface of the proximal portion of the enclosure, the adhesive patch including an opening for the metal disc and a bio-compatible adhesive on a proximal surface for removably attaching the device to the skin of the user.
